(19) United States
(12) Patent Application Publication
(10) Pub. No.: US 2023/0048035 A1
(43) Pub. Date: Feb. 16, 2023

(54) Rudbeckia plant 'RDSGO01-0'

(71) Applicant: GreenFuse Botanicals, Santa Paula, CA (US)

(72) Inventor: James Devereux, Fort Collins, CO (US)

(73) Assignee: GreenFuse Botanicals, Santa Paula, CA (US)

(21) Appl. No.: 17/401,106

(22) Filed: Aug. 12, 2021

(51) Int. Cl. A01H 6/14; A01H 5/02

(52) U.S. Cl. CPC A01H 6/14; A01H 5/02

(57) ABSTRACT

The invention relates to the field of Rudbeckia hirta, specifically, the variety designated ‘RDSGO01-0’. The variety ‘RDSGO01-0’ is characterized by day-length neutral flowering, cold tolerance to USDA Zone 5 and abundant flowering of single infloresences with golden yellow ray florets. The present invention relates to plant parts, including cells and any propagative material of the new variety ‘RDSGO01-0’, and use of any of the plant parts for reproducing the new variety ‘RDSGO01-0’. The present invention relates to methods using any plant parts or progeny of ‘RDSGO01-0’ for the purpose of deriving additional new Rudbeckia hirta varieties. The present invention relates to seed, plants and plant parts produced by crossing ‘RDSGO01-0’ with any other Rudbeckia hirta variety or other plant. The present invention also relates to methods to produce new varieties of Rudbeckia hirta oleracea using the variety ‘RDSGO01-0’ and applying plant breeding techniques.

### FIELD OF THE INVENTION

The present invention relates to a new variety of Rudbeckia hirta referred to as ‘RDSGO01-0’ as well as to new, distinct and stable characteristics found in Rudbeckia hirta. The present invention relates to plants which have all of the morphological and physiological characteristics described herein, as well as plant parts which can be used to reproduce plants having the characteristics specific to Rudbeckia hirta referred to as ‘RDSGO01-0’ . The present invention also relates to methods for producing these plants of Rudbeckia hirta exhibiting the characteristics described herein. Furthermore, the present invention relates to a method of producing progeny Rudbeckia hirta plants by crossing the Rudbeckia hirta oleracea referred to as ‘RDSGO01-0’, as either the female or seed or male or pollen parent, with another Rudbeckia hirta plant, or ‘RDSGO01-0’, and selecting progeny. The present invention also relates to methods to produce new varieties of Rudbeckia hirta using the variety ‘RDSGO01-0’ in a breeding program.

### BACKGROUND OF THE INVENTION

The present invention relates to a new, distinct and stable variety of Rudbeckia hirta, hereinafter referred to by the variety denomination ‘RDSGO01-0’. The new Rudbeckia hirta ‘RDSGO01-0’ originated from a controlled breeding program in Santa Paula, Calif.

Rudbeckia hirta belong to the family Asteraceae a broad Genus consisting of more than 30,000 species. Rudbeckia hirta is native to Eastern and Central North America. Rudbeckia hirta is a flowering perennial with increasing popularity. Hative bees as well as butterflies are known to be attracted to the flower.

Methods for propagation are well known and include vegetative cuttings, tissue culture and less commonly division of plants. Rudbeckia hirta propagates readily from seed, however clonal stability is not assured with seed propagation.

Varieties of Rudbeckia hirta, are propagated both by sexual(seed) and asexual (vegetative) means are available on the market. These varieties are primarily considered annuals, as plants are unlikely to return after one year. Plants may seed freely, creating the impression a plant has returned. However, these are not identical clones, but annual seedlings, which may have variable phenotypes.

Rudbeckia hirta is a short-lived perennial, recommended to be treated as an annual by home gardeners. Jeffers, A. (2017), Rudbeckia Factsheet HGIC 1192, Clemson University. The new variety disclosed herein ‘RDSGO01-0’, is a true perennial to USDA Zone 5. Individual plants of ‘RDSGO01-0’ have been observed to survive unprotected outdoors during Winters in Colorado in confidential trials.

Most, if not all known commercial varieties of Rudbeckia hirta bloom under long day conditions. As consumers prefer to buy plants when in flower, this limits the sales period to late Spring and Summer, unless artificial light conditions are provided to plants.

The new variety begins blooming under any day length condition. The day-length neutral characteristic increases the sales season, without the need to manipulate the photoperiod.

The variety described herein is an ornamental Rudbeckia hirta plant, having the above-mentioned improvements combined with attractive ornamental characteristics. Plants retain the desirable ornamental traits of Rudbeckia hirta, including large, colorful inflorescences, remontant flowering, together with novel and improved floral induction.

To those trained in the art, the improvements described herein will be found both valuable and quantitative assets.

### SUMMARY OF THE INVENTION

The following embodiments and aspects thereof are described in conjunction with systems, tools and methods which are meant to be exemplary, not limiting in scope.

The present invention provides Rudbeckia hirta plant selections which begin flowering under any day-length, being day-length neutral. Plants of Rudbeckia hirta ‘RDSGO01-0’ are hardy to USDA Zone 5. These qualities, combined with the description included herein, distinguish the new cultivar from known Rudbeckia hirta varieties.

These and other improvement and objectives have been achieved in accordance with the present invention which provide ‘RDSGO01-0’ as a new Rudbeckia hirta cultivar that is the product of a planned breeding program conducted by the inventor in Santa Paula, Calif.

The new variety ‘RDSGO01-0’ can be produced by asexual reproduction to produce a population of progeny plants, each of which has the combination of characteristics as herein disclosed for the new variety ‘RDSGO01-0’.

### DEFINITIONS

Terminology below may be useful for description and understanding of the new invention.
Day-Length: A common term referring to the hours of daylight required to induce and develop flowering. Syn. photoperiodism.
Day-Length Neutral: A flowering condition in which neither short nor long days are required to induce and develop flowering.
Dormant: Temporarily inactive.
Morphology: Form and structure.
Plant form: Overall structure and shape of the plant.
Progeny: Off-spring or descendants.

Additional Terminology, Related to Polyploidy
Euploids—polyploids with exact multiples of the complete set of chromosomes specific to a species. Haploid or Monoploid (one set; 1×), Diploid (two sets; 2×), Triploid (three sets; 3×), Tetraploid (four sets; 4×), Pentaploid (five sets; 5×), Hexaploid (six sets; 6×), Octaploid (eight sets; 8×), Decaploid (ten sets; 10×), Dodecaploid (12 sets; 12×).
Autopolyploids—polyploids with multiple chromosome sets derived from a single species—usually derived from chromosome doubling.
Allopolyploids—polyploids whose chromosomes are derived from different species. are a combination of genomes of different species (Acquaah 2007) due to hybridisation of two or more genomes followed by chromosome doubling or by the fusion of unreduced gametes between species (Acquaah 2007; Chen 2010; Jones et al. 2008; Ramsey and Schemske 1998). Can accure naturally or artificially
Aneuploids—polyploids that contain either an addition or deletion of one or more specific chromosome(s) to the total number of chromosomes that usually make up the ploidy of a species (Acquaah 2007; Ramsey and Schemske 1998). Aneuploids are formed due to the formation of univalents and multivalents during meiosis of euploids

### DEPOSIT INFORMATION

Biological material which is the variety ‘RDSGO01-0’ is to be deposited with Bigelow Laboratory for Ocean Sciences, East Boothbay, Me., a Budapest Treaty recognized depository which affords permanence of the deposit. Upon receipt the accorded International Depository Authority Accession Number will be provided as necessary.

### BRIEF DESCRIPTION OF THE DRAWINGS

The patent or application file contains multiple drawings executed in color. Copies of this patent or patent application publication with color drawing will be provided by the Office upon request and payment of the necessary fees.

The accompanying photographs illustrate the overall appearance and morphology of the new Rudbeckia hirta ‘RDSGO01-0’ showing the colors as true as is reasonably possible with colored reproductions of this type. Colors in the photograph may differ slightly from the color values cited in the detailed botanical description which accurately describes the color of ‘RDSGO01-0’.

FIG. 1 illustrates a side view of plants of Rudbeckia hirta ‘RDSGO01-0’ at approximately 12 to 13 weeks of age. This photo was taken in Santa Paula, Calif.

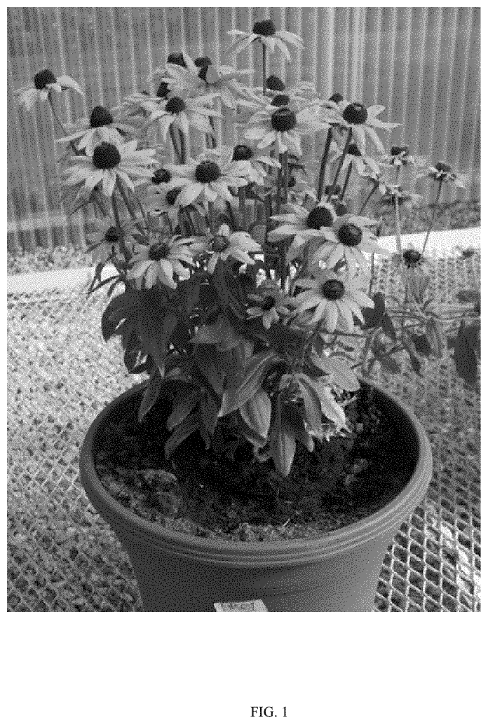

FIG. 2 illustrates a close-up view of an inflorescence of Rudbeckia hirta ‘RDSGO01-0’

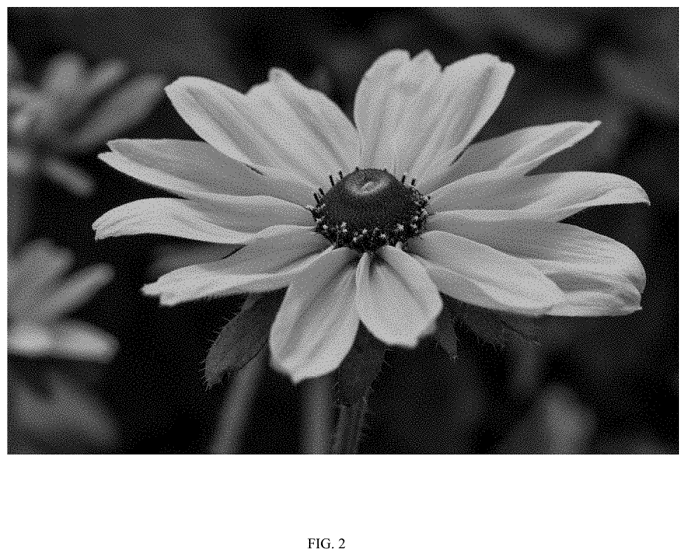

### OBJECTS OF THE INVENTION

The following embodiments and aspects thereof are described in conjunction with system, tools and methods which are meant to be exemplary, not limiting in scope

The present invention relates to Rudbeckia hirta plants, and parts thereof, having all the physiological and morphological characteristics of Rudbeckia hirta ‘RDSGO01-0’.

Another embodiment relates to a plant produced by vegetative means which are Rudbeckia hirta ‘RDSGO01-0’. The present invention also relates to plant parts, such as pollen, seeds or inflorescence produced by Rudbeckia hirta ‘RDSGO01-0’.

Another embodiment relates to a method of producing seed which are Rudbeckia hirta ‘RDSGO01-0’.

Another embodiment also relates to a method of producing Rudbeckia hirta progeny comprising the steps of (a) self-pollinating Rudbeckia hirta ‘RDSGO01-0’ (b) harvesting seeds produced from said self-pollination; and (c) producing plants from said harvested seeds.

The present invention also relates to producing progeny plants from the cross of Rudbeckia hirta ‘RDSGO01-0’, as the female or male parent, with another Rudbeckia hirta plant, and selecting progeny plants from this cross.

The present invention also relates to producing progeny plants of Rudbeckia hirta ‘RDSGO01-0’, by any known means of vegetative propagation.

The present invention also relates to producing progeny plants from Rudbeckia hirta ‘RDSGO01-0’, from natural or induced mutation.

Another embodiment relates to tissue culture produced from protoplast of cells from Rudbeckia hirta ‘RDSGO01-0’ plants disclosed in the subject application, wherein said cells or protoplasts are produced from a plant part selected from the group consisting of pollen, ovules, embryos, protoplasts, meristematic cells, callus, leaves, anthers, cotyledons, hypcotyl, pistils, roots, root tips, flowers, seeds, petiole and stems.

Another embodiment relates to a plant or a part thereof, produced by growing Rudbeckia hirta ‘RDSGO01-0’, wherein the plant part comprises at least one cell of Rudbeckia hirta ‘RDSGO01-0’.

Another embodiment relates to tissue or cell culture of regenerable cells produced from the plants of Rudbeckia hirta ‘RDSGO01-0’ as well as an Rudbeckia hirta plant regenerated from the tissue or cell culture of Rudbeckia hirta ‘RDSGO01-0’.

Another embodiment relates to a method of vegetatively propagating the plant of Rudbeckia hirta ‘RDSGO01-0’, comprising the steps of: collecting tissue or cells capable of being propagated from a plant of Rudbeckia hirta ‘RDSGO01-0’; cultivating said tissue or cells to obtain proliferated shoots; and rooted said shoots to obtain rooted plantlets; or cultivating said tissue or cells to obtain shoots or to obtain plantlets and a plant produced by growing the plantlets or shoots of said plant.

A further embodiment relates to a method for developing an Rudbeckia hirta plant in an Rudbeckia hirta breeding program, comprising applying plant breeding techniques comprising crossing, recurrent selection, mutation breeding, wherein said mutation breeding selects for a mutation that is spontaneous or artificially induced, backcrossing, pedigree breeding, marker enhanced selection, haploid/double haploid production, ploidy manipulation, or transformation to the Rudbeckia hirta plant of Rudbeckia hirta ‘RDSGO01-0’, or its parts, wherein application of said techniques results in development of an Rudbeckia hirta plant.

A further embodiment relates to a method of introducing a mutation into the genome of Rudbeckia hirta ‘RDSGO01-0’, and wherein the resulting plant comprises at least one genome mutation and producing plants there from.

In addition to the exemplary aspects and embodiments described above, further aspects and embodiments will become apparent by study of the following descriptions.

### BRIEF DESCRIPTION OF THE INVENTION

This invention is directed to Rudbeckia hirta plants having all the morphological and physiological characteristics of the variety ‘RDSGO01-0’ produced from either vegetative cuttings or tissue culture.

The new Rudbeckia hirta ‘RDSGO01-0’ can be produced by asexually reproducing progeny. Asexual reproduction of the new cultivar by vegetative means was first performed by vegetative cuttings during May of 2018, in Santa Paula, Calif. The first ‘RDSGO01-0’ plants propagated through the use of such cuttings are maintained in Santa Paula, Calif. and have reproduced multiple generations. Subsequent asexual reproduction has demonstrated that the new cultivar reproduces true-to-type and that the combination of characteristics as herein disclosed for the new cultivar are firmly fixed and retained through successive generations of asexual reproduction.

The following traits have been repeatedly observed and are determined to be unique characteristics of ‘RDSGO01-0’ which in combination distinguish these Rudbeckia hirta as new and distinct cultivars:
1. Abundant flowering.
2. Flowering is day length neutral and plants can flower year round.
3. Cold tolerant to the roots to USDA Zone 5, individual plants survive and return as a perennial.
4. Single terminal capitulum having golden yellow ray florets and a deep brown disc.

### COMMERCIAL COMPARISON

‘RDSGO01-0’ can be compared to the commercially known variety Rudbeckia hirta ‘Balrudosh’, USPP 25,496. These plants are similar in most horticultural characteristics. However, plants of the new cultivar ‘RDSGO01-0’ differ in the following:

1. ‘Baldorush’ has double-type inflorescences; ‘RDSGO01-0’ has single inflorescences.
2. ‘Baldorush’ has streaked ray florets; ‘RDSGO01-0’ has solid-colored ray florets.
3. ‘Baldorush’ requires long days to flower; ‘RDSGO01-0’ will flower under any day length.

‘RDSGO01-0’ can also be compared to the commercially known variety Rudbeckia hirta ‘RUDHT37’, USPP 30,657. These plants are similar in most horticultural characteristics. However, plants of the new cultivar ‘RDSGO01-0’ differ in the following ways:

1. ‘RUDHT37’ has a broad, upright habit; ‘RDSGO01-0’ is more upright than broad in habit.
2. ‘RUDHT37’ has large single inflorescences about 5″ to 6″ in diameter; flowers of ‘RDSGO01-0’ are typically smaller, 3″ to 5″ in diameter.
3. ‘RUDHT37’ requires long days to flower; ‘RDSGO01-0’ will flower under any day length.

‘RDSGO01-0’ has not been tested and observed under all possible environmental conditions. The phenotype of the new cultivar may vary with variations in environment such as temperature, light intensity, frequency of fertilization, composition of fertilizer, flowering treatment, day length and humidity, without any change in the genotype of the plant.

For example, substantial differences in plant height and diameter can be observed, depending upon environmental conditions and planting density. Inflorescence size and color can vary as well.

### DETAILED BOTANICAL DESCRIPTION

The aforementioned drawings, together with the following observations, measurements and values describe the new Rudbeckia hirta ‘RDSGO01-0’ as grown in a greenhouse in Santa Paula, Calif. Plants of ‘RDSGO01-0’ were grown in a research greenhouse with temperatures ranging from approximately 5° C. to 18° C. during the day and night temperatures ranging from approximately 20° C. to 30° C. in the day and approximately 15° C. to 25° C. in the day. No artificial lighting or photoperiodic treatments were conducted. Plants were measured at approximately 4.5 months old from an unrooted cutting.

Color reference are made to the Royal Horticultural Society Colour Chart (RHS), 2007 edition, except where general colors of ordinary significance are used. Color values were taken under daylight conditions in a greenhouse in Ventura County, Calif.

### PLANT

Growth Habit: Hardy, upright, herbaceous perennial.
Height: To top of foliar plane: Approximately 38 cm.
To top of flowering plane: Approximately 60 cm.
Plant Spread: Approximately 25 cm.
Growth Rate: Moderate
Branching Characteristics: Basal and low lateral branching. 2 to 4 branches from a pinch.
Length of Branches: Approximately 12 to 18 cm, excluding peduncle.
Diameter Branches: Approximately 5 to 8 mm.
Quantity of Branches: Approximately 4 to 6 basal branches, each basal branch with 2 2 to 4 lateral branches.
Texture of Lateral Branches: Hispid. Hairs about 4 mm long, colored near White 155A
Color of Lateral Branches: Near RHS Green 143A. Internodes and region near 5 internodes flushed Greyed-Purple 183B.
Aspect: Approximately 10° to 15° angle from base of plant.
Strength of Lateral Branches: Very strong.
Internode length: Average range 4.0 to 7.5 cm.
Age of Plant Described: Approximately 4 to 5 months old.

### FOLIAGE

Leaf:
   Arrangement: Flowering stems produce sub-opposite or alternate occurring foliage. Basal foliage occurs in irregular whorls.
   Average Length: Average range 9 to 13 cm.
   Average Width: Average range 3.5 to 4.5 cm.
   Shape of blade: Elliptic.
   Apex: Acute.
   Base: Attenuate.
   Attachment: Sessile
   Margin: Entire to sparsely and shallowly serrate.
   Texture of top surface: Densely pilose to nearly hirsute, hairs stiff, but, soft.
   Average hair length approximately 3 mm, colored near RHS White 155A.
   Texture of bottom surface: Densely pilose to nearly hirsute, hairs stiff, but, soft.
   Average hair length approximately 2 mm, colored near RHS White 155A.
Color:
   Young foliage upper side: Near RHS Green 137A.
   Young foliage under side: Near RHS Green 138A.
   Mature foliage upper side: Near RHS Green 137A.
   Mature foliage under side: Near RHS Green 137C.
Venation:
   Type: Pinnate
   Venation color upper side: Near RHS Yellow-Green 145C.
   Venation color under side: Near RHS Yellow-Green 145C.

### FLOWER

Natural flowering season: Day length neutral, flowering begins approximately 6 weeks after planting during any time of the year.
Bud:
   Bud shape: Globular.
   Bud height: Approximately 13 mm.
   Bud diameter: Approximately 15 mm.
   Bud color: Near Yellow-Green 144C.
Inflorescence:
   Form: Terminal capitulum consisting of numerous disc florets, one or two rows of ray florets and two to three rows of involucral bracts.
Flower:
   Diameter of entire flower. Average range 6 to 8 cm.
   Depth of flower: Approximately 2.0 cm.
   Disc diameter: Approximately 2.5 cm.
   Disc depth: Approximately 2.0 cm at maturity.
   Receptacle shape: Conical.
   Receptacle height: Average 1 cm.
   Receptacle diameter: Average 1.3 cm at base, apex near 5 mm.
   Receptacle Color: Near RHS White 155A.
   Quantity of inflorescences per lateral stem: 1
   Quantity of open inflorescence and buds per plant: Average 20 to 40 fully open flowers, and 10 to 14 buds.
   Fragrance: Faint earthy scent.
Ray Florets:
   Arrangement: Rotate, single to semi-double row.
   Shape: Elliptic.
   Number of Ray Florets: Average range 15 to 18.
   Length: Approximately 3.5 to 4.5 cm.
   Width: Approximately 1.5 cm.
   Apex shape: Round, or 2 to 6 irregular shallow lobes, with round apices.
   Base: Attenuate.
   Margin: Entire.
   Texture all surfaces: Smooth, matte, ribbed lengthwise.
Color:

Ray Florets
Upper surface at first opening: Near RHS Yellow-Orange 14A.
Upper surface at maturity: Near RHS Yellow-Orange 17C.
Under surface at first opening: Near RHS Yellow 13A.
Under surface at maturity: Near RHS Yellow-Orange 17C.

Disc Florets
Number of Disc Florets: Approximately 300.
Arrangement: Acropetally on disc.
Length: Approximately 5 to 7 mm
Width: Approximately 1 mm.
Shape: Tubular
Margin: Entire.
Apex: Rounded when immature, serrate with age.
Base: Fused
Texture: Matte.
Color Disc Florets:
   At first opening: Near RHS Greyed-Purple 187A, base near Yellow 10D.
   At maturity: Near RHS Brown 200B, base near White N155C.
   Fading: Near RHS Brown 200B.
Phyllaries/Involucal Bracts
   Arrangement. Rotate, in 2 or 3 rows.
   Number: Approximately 25
   Length: Average range 7 to 24 mm
   Width: Approximately 4 to 7 mm
   Shape: Lanceolate.
   Apex: Acute.
   Base: Truncate, fused.
   Margin: Entire, ciliate.
   Texture: Sericeous upper and lower surfaces.
   Color:
      Inside color near RHS Green 137A.
      Outside color near RHS Green 137B.

### PEDUNCLE

Length: Average range 20 to 32 cm.
Width: Average 5 mm.
Texture: Hispid
Color: Near RHS Green 137A
Strength: Very strong.

### REPRODUCTIVE ORGANS

Ray Florets:
Gynoecium: Not observed
Androecium: Not present.
Disc Florets:
Androecium:
Stamen number: 2 to 4

Filament length: Average 2-3 mm

Filament color: Near Greyed-Purple N186C.

Anther shape: Forked/curled

Anther length: 0.5 mm

Anther color: Greyed-Purple N186C.

Pollen amount: Moderate

Pollen color: Near Yellow-Orange 14C
Gynoecium:
Pistil length: Approximately 4 mm.
Stigma shape: Decurrent.
Stigma color: Near RHS Greyed-Purple N186C.
Style length: About 3 mm
Style color: Lower section near Yellow 10D upper section near Greyed-Purple N186C
Ovary color: Near RHS Yellow 10D

### OTHER CHARACTERISTICS

Seeds and fruits: Not observed to date.
Disease/pest resistance: Neither resistance nor susceptibility to normal diseases and pests of Rudbeckia hirta have been observed.
Temperature tolerance: Roots are low temperature tolerant to USDA Zones 5. Individual plants(clones) are perennial.

We claim:

1. Rudbeckia hirta ‘RDSGO01-0’, representative biological material to be deposited at Bigelow Laboratory for Ocean Sciences, East Boothbay, Me.

2. A plant or a plant part thereof produced by growing the plant of claim 1, wherein the plant or plant part comprises at least one cell of Rudbeckia hirta ‘RDSGO01-0’.

3. A Rudbeckia hirta plant or part thereof, having the physiological and morphological characteristics of the plants of claim 1.

4. The tissue or cell culture of regenerable cells produced from the plant of claim 1.

5. The tissue or cell culture of claim 4, comprising tissues or cells from a plant part selected from the group consisting of leaves, vegetative cuttings, pollen, embryos, cotyledons, hypocotyl, meristematic cells, roots, root tips, pistils, anthers, flowers and stems.

6. A method of producing Rudbeckia hirta progeny comprising the steps of
(a) crossing a plant of Rudbeckia hirta ‘RDSGO01-0’ as a female or male parent with another Rudbeckia hirta plant or other plant and (b) selecting progeny.

7. The method according to claim 6, wherein the second plant is Rudbeckia hirta ‘RDSGO01-0’.

8. (canceled)

9. A method for developing a Rudbeckia hirta plant in a plant breeding program using plant breeding techniques, including crossing, recurrent selection, mutation breeding, wherein said mutation breeding selects for a mutation that is spontaneously or naturally induced or artificially induced, backcrossing, pedigree breeding, marker enhanced selection, haploid/double haploid production or transformation to a plant of Rudbeckia hirta ‘RDSGO01-0’, or it's parts, or progeny wherein application of said techniques results in development of a Rudbeckia hirta plant.
